(19) United States
(12) Patent Application Publication
(10) Pub. No.: US 2025/0032694 A1
(43) Pub. Date: Jan. 30, 2025

(54) ADSORBENT MATERIAL, ADSORBENT SHEET, SEPARATION MEMBRANE, ARTIFICIAL DIALYSIS MACHINE, AND PRODUCTION METHOD

(71) Applicant: Murata Manufacturing Co., Ltd., Nagaokakyo-shi (JP)

(72) Inventors: Yuki KIMURA, Nagaokakyo-shi (JP); Ichitaro Okamura, Nagaokakyo-shi (JP); Takeshi Torita, Nagaokakyo-shi (JP); Yusuke Kobayashi, Yokohama-shi (JP); Hiromichi Wakui, Yokohama-shi (JP); Kengo Azushima, Yokohama-shi (JP); Kouichi Tamura, Yokohama-shi (JP)

(21) Appl. No.: 18/915,818

(22) Filed: Oct. 15, 2024

Related U.S. Application Data
(63) Continuation of application No. PCT/JP2023/015824, filed on Apr. 20, 2023.
(63) Continuation of application No. PCT/JP2022/037909, filed on Oct. 11, 2022.

(30) Foreign Application Priority Data
Apr. 21, 2022 (JP) 2022-070348
Jul. 13, 2022 (JP) 2022-112701
Oct. 11, 2022 (JP) 2022-163375

(51) Int. Cl. A61M 1/36

(52) U.S. Cl. CPC A61M 1/3679; A61M 1/3687; A61M 2202/07; A61M 2202/20; A61M 2207/00

(57) ABSTRACT

An adsorbent material used for adsorbing a disease-causing substance, the adsorbent material including two-dimensional particles having one or plural layers, the one or plural layers containing a layer body represented by: MₘXₙ, wherein M is at least one metal of Group 3, 4, 5, 6, or 7, X is a carbon atom, a nitrogen atom, or a combination thereof, n is not less than 1 and not more than 4, m is more than n but not more than 5, and a modifier or terminal T exists on a surface of the layer body, wherein T is at least one selected from a hydroxyl group, a fluorine atom, a chlorine atom, an oxygen atom, and a hydrogen atom.

### CROSS REFERENCE TO RELATED APPLICATIONS

The present application is a continuation of International application No. PCT/JP2023/015824, filed Apr. 20, 2023, which claims priority to Japanese Patent Application No. 2022-070348, filed Apr. 21, 2022, Japanese Patent Application No. 2022-112701, filed Jul. 13, 2022, International application No. PCT/JP2022/037909, filed Oct. 11, 2022, and Japanese Patent Application No. 2022-163375, filed Oct. 11, 2022; and is a continuation of International application No. PCT/JP2022/037909, filed Oct. 11, 2022, which claims priority to Japanese Patent Application No. 2022-070348, filed Apr. 21, 2022; the entire contents of each of which are incorporated herein by reference.

### TECHNICAL FIELD

The present disclosure relates to an adsorbent material, an adsorbent sheet, a separation membrane, an artificial dialysis machine, and a production method.

### BACKGROUND ART

The number of patients with renal disease is on an increasing trend year by year, and according to the statistics of the Japanese Society for Dialysis Therapy, the number of chronic dialysis patients has increased by approximately 17% from 2010 to 2020. When the renal function decreases due to renal disease, the disease-causing substances accumulate in the blood, and as a result, uremia, electrolyte imbalance, autoimmune disease, and the like can be caused. In order to treat such various symptoms associated with renal disease, a hemodialysis type renal function substitute or the like that removes a disease-causing substance to the outside of the body is used.
Non-Patent Document 1 describes an adsorption type blood purifier using cellulose beads on which cetylamine is immobilized as an adsorbing body as a filter used for a hemodialysis type renal function substitute.
Non-patent Document 1: The package insert of medical devices “Lixelle”, KANEKA CORPORATION, revised in January 2017 (10 edition)

### SUMMARY OF THE DISCLOSURE

In the adsorbent material described in Non-patent Document 1, the adsorbate is limited to certain materials.

One object of the present disclosure is to provide a novel adsorbent material used for adsorbing a wider range of adsorbates as compared to Non-patent Document 1. Another object of the present disclosure is to provide an adsorbent sheet using such an adsorbent material, a separation membrane, an artificial dialysis machine, and a method for producing such an adsorbent.

The adsorbent material of the present disclosure comprises two-dimensional particles having one or plural layers, wherein the one or plural layers comprise a layer body represented by:
MₘXₙ
wherein M is at least one metal of Group 3, 4, 5, 6, or 7, X is a carbon atom, a nitrogen atom, or a combination thereof, n is not less than 1 and not more than 4, m is more than n but not more than 5, and a modifier or terminal T existing on a surface of the layer body, wherein T is at least one selected from a hydroxyl group, a fluorine atom, a chlorine atom, an oxygen atom, and a hydrogen atom, and
the adsorbent material is used for adsorbing a disease-causing substance.

The present disclosure provides a novel adsorbent material used for adsorbing a wider range of adsorbates as compared with Non-patent Document 1. In addition, the present disclosure provides an adsorbent sheet using an adsorbent material used for adsorbing a wider range of adsorbates than in Non-patent Document 1, a separation membrane, an artificial dialysis machine, and a method for producing such an adsorbent material.

### BRIEF DESCRIPTION OF THE DRAWINGS

FIGS. 1(a) and 1(b) are schematic cross-sectional views showing two-dimensional particles that are a layered material usable for an adsorbent material of the present disclosure, in which FIG. 1(a) shows a single-layer MXene, and FIG. 1(b) shows a multilayer (exemplarily, two layers) MXene.

FIG. 2 is a diagram schematically illustrating an example of an artificial dialysis machine using the adsorbent material according to the present disclosure.

FIG. 3 is a diagram schematically illustrating another example of an artificial dialysis machine using an adsorbent material according to the present disclosure.

### DETAILED DESCRIPTION OF THE PREFERRED EMBODIMENTS

### Embodiment 1: Adsorbent Material

The adsorbent material of the present disclosure is used for adsorbing a disease-causing substance, and the adsorbent material includes two-dimensional particles having one or plural layers, wherein the one or plural layers comprise a layer body represented by a composition formula below:

MₘXₙ
wherein M is at least one metal of Group 3, 4, 5, 6, or 7, X is a carbon atom, a nitrogen atom, or a combination thereof, n is not less than 1 and not more than 4, m is more than n but not more than 5, and a modifier or terminal T existing on a surface of the layer body, wherein T is at least one selected from the group consisting of a hydroxyl group, a fluorine atom, a chlorine atom, an oxygen atom, and a hydrogen atom.

The present disclosure provides an adsorbent material capable of adsorbing various adsorbates, and particularly, provides an adsorbent material capable of adsorbing a disease-causing substance. Although the present disclosure should not be construed as being limited to a specific theory, the reason why the adsorbent material of the present disclosure adsorbs various adsorbates is considered as follows.

The two-dimensional particles contained in the adsorbent material of the present disclosure are particles of a layered material also referred to as MXene, and have one or more modifier or terminal T (T is at least one selected from the group consisting of a hydroxyl group, a fluorine atom, a chlorine atom, an oxygen atom, and a hydrogen atom) on the surface of the layer body represented by MₘXₙ. With such a configuration, a charged site presents in the layer included in the two-dimensional particle, and the layer surface of the two-dimensional particle usually is negatively charged. In addition, the end surface of the two-dimensional particle (the peripheral edge of the layer of the two-dimensional particle) is usually positively charged. Therefore, when viewed as the entire two-dimensional particle, the positively charged adsorbate (for example, a cation or the like) tends to be adsorbed between the layers, and the negatively charged adsorbate (for example, an anion or the like) tends to be adsorbed on the end face of the layer.

The inventors of the present disclosure have studied an adsorbates which the two-dimensional particles are capable of adsorbing, and found that the two-dimensional particles adsorb a relatively high molecular weight compound such as a protein in addition to the positively or negatively charged adsorbent.

The two-dimensional particle may be understood as a layered material or layered compound, also denoted “MₘXₙTₛ”, wherein s is any number, and conventionally x or z may be used instead of s. Typically, n can be 1, 2, 3, or 4, but is not limited thereto.

In the present disclosure, the layer may be referred to as an MXene layer, and the two-dimensional particle may be referred to as an MXene two-dimensional particle or an MXene particle.

In the above formula of MXene, M is at least one selected from the group consisting of Ti, Zr, Hf, V, Nb, Ta, Cr, Mo, and Mn, and preferably contains at least Ti, and more preferably contains at least one selected from the group consisting of Ti, V, Cr, and Mo, and contains at least Ti.

The ratio of Ti atoms in M may be preferably not less than 50 atom % and not more than 100 atom % or less, more preferably not less than 70 atom % and not more than 100 atom % or less, and still more preferably not less than 90 atom % and not more than 100 atom % or less.

MXenes whose above formula MₘXₙ is expressed as below are known:

Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti, V)₂C, (Ti, Nb)₂C, W₂C, W_{1.3}C, Mo₂N, Nb_{1.3}C, Mo_{1.3}Y_{0.6}C (in the above formula, “1.3” and “0.6” mean about 1.3 (=4/3) and about 0.6 (=⅔), respectively.),
Ti₃C₂, Ti₃N₂, Ti₃ (CN), Zr₃C₂, (Ti, V)₃C₂, (Ti₂Nb)C₂, (Ti₂Ta)C₂, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr)C₂, (Mo₂Hf)C₂, (Mo₂V)C₂, (Mo₂Nb)C₂, (Mo₂Ta)C₂, (W₂Ti)C₂, (W₂Zr)C₂, (W₂Hf)C₂, or
Ti₄N₃, V₄C₃, Nb₄C₃, Ta₄C₃, (Ti, Nb)₄C₃, (Nb, Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, (W₂Hf₂)C₃, (Mo_{2.7}V_{1.3})C₃ (in the above formula, “2.7” and “1.3” mean about 2.7 (=8/3) and about 1.3 (=4/3), respectively.)

Typically, in the above formula, M may comprise Ti and X may be a carbon atom or a nitrogen atom, preferably M may be Ti and X may be a carbon atom. For example, the MAX phase is Ti₃AlC₂ and MXene is Ti₃C₂Tₛ (in other words, M is Ti, X is C, n is 2, and m is 3).

In the present disclosure, MXene may contain a relatively small amount of A atoms derived from the MAX phase of the precursor, for example, 10 mass % or less with respect to the original A atoms. The remaining amount of A atoms can be preferably 8% by mass or less, and more preferably 6% by mass or less. However, even if the residual amount of A atoms exceeds 10 mass %, there may be no problem depending on the application and use conditions of the two-dimensional particles.

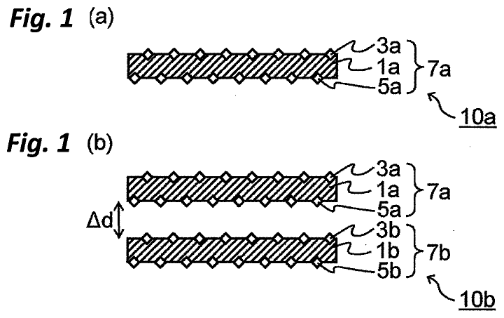

The two-dimensional particles are an aggregate containing MXene particles (hereinafter, simply referred to as “MXene particles”)10a (single-layer MXene particles) of one layer schematically exemplified in FIG. 1(a). The MXene particle 10a is more specifically the MXene layer 7a having a layer body (MₘXₙ layer) 1a represented by MₘXₙ and modifier or terminal T3a, 5a presenting on the surface of the layer body 1a (more specifically, at least one of two surfaces facing each other in each layer). Therefore, the MXene layer 7a is also represented as “MₘXₙTₛ”, and s is an arbitrary number.

The two-dimensional particle may include one or plural layers. Examples of the MXene particles (multilayer MXene particles) of the plurality of layers include, but are not limited to, the MXene particle 10b of two layers as schematically shown in FIG. 1(b). 1b, 3b, 5b, and 7b in FIG. 1(b) are the same as 1a, 3a, 5a, and 7a in FIG. 1(a) described above. In the case of the multilayer MXene particles, two adjacent MXene layer (e.g., 7a and 7b) may not necessarily be completely separated from each other, but may be partially in contact with each other. The MXene particle 10a may exist as a single-layer resulting from separating MXene particle 10b layer by layer, and may be a mixture of the single-layer MXene particle 10a and the multilayer MXene particle 10b.

Although the present embodiment is not limited, the thickness of each layer contained in the MXene particles (which corresponds to the MXene layers 7a, 7b) can be, for example, not less than 0.8 nm and not more than 5 nm, particularly not less than 0.8 nm and not more than 3 nm (it may vary mainly depending on the number of M atomic layers included in each layer). For individual layered bodies of multilayer MXene particles that may be included, the interlayer distance (Alternatively, the void dimension is indicated by Δd in FIG. 1(b).) may be, for example, not less than 0.8 nm and not more than 10 nm, particularly not less than 0.8 nm and not more than 5 nm, more particularly about 1 nm, and the total number of layers may be not less than 2 and not more than 20,000.

In one aspect, the ratio of (the average value of the major diameter of the two-dimensional surfaces of the two-dimensional particles)/(the average value of the thicknesses of the two-dimensional particles) is 1.2 or more, preferably 1.5 or more, and more preferably 2 or more. The average value of the major diameters of the two-dimensional surfaces of the two-dimensional particles and the average value of the thicknesses of the two-dimensional particles may be obtained by a method described later.

In one aspect, in the two-dimensional particles of the present embodiment the multilayer MXene particle that can be contained comprises a two-dimensional particles having a small number of layers obtained through a delamination treatment. The “small number of layers” means that, for example, the number of laminated MXene layers is six or less. In addition, the thickness of the multilayer MXene particle having a small number of layers in the lamination direction is preferably 15 nm or less, and more preferably 10 nm or less. Hereinafter, the “multilayer MXene particles having a small number of layers” may be referred to as “few-layer MXene particles”. The single-layer MXene particles and the few-layer MXene particles may be collectively referred to as “single-layer/few-layer MXene particles”. By containing the single-layer/few-layer MXene particles, the conductivity of the resulting membrane may increase.

In the multilayer MXene particle having a small number of layers, the ratio of (the average value of the major diameters of the two-dimensional surfaces of the two-dimensional particles)/(the average value of the thicknesses of the two-dimensional particles) is 1.2 or more, preferably not less than 1.5 and not more than 10, and more preferably not less than 2 and not more than 5. Hereinafter, the “MXene particles having a small number of layers” may be referred to as “few-layer MXene particles”. The single-layer MXene particles and the few-layer MXene particles may be collectively referred to as “single-layer/few-layer MXene particles”. As a result, the membrane formability of the membrane containing the two-dimensional particles can be improved.

Examples of the single-layer/few-layer MXene particles include two-dimensional particles obtained through delamination treatment.

In one aspect, the two-dimensional particles of the present embodiment preferably contains single-layer MXene particles and few-layer MXene particles, that is, single-layer/few-layer MXene particles. In the two-dimensional particles of the present embodiment, the proportion of the single-layer/few-layer MXene particles having a thickness of 15 nm or less is preferably 90 vol % or more, more preferably 95 vol % or more. As a result, the membrane formability of the membrane containing the two-dimensional particles can be improved.

### (Average Value of Major Diameters of Two-Dimensional Surfaces of Two-Dimensional Particles)

In the two-dimensional particles of the present embodiment, the average value of the major diameters of the two-dimensional surfaces is not less than 0.01 μm and not more than 20 km. Hereinafter, the average value of the major diameters of the two-dimensional surfaces may be referred to as “average flake size”.

The larger the average flake size, the better the orientation of the two-dimensional particles in the material containing the two-dimensional particles. The orientation of the two-dimensional particles can be evaluated by, for example, the conductivity of the material containing the two-dimensional particles.

The average value of the major diameters of the two-dimensional surfaces is preferably 0.02 μm or more, and more preferably 0.05 μm or more. When the delamination treatment of MXene is performed by subjecting MXene to the ultrasonic treatment, most of MXene is reduced in diameter to about several hundred nanometers in terms of major diameter by the ultrasonic treatment, so that the membrane formed of the single-layer MXene delaminated by the ultrasonic treatment is considered to have low orientation of two-dimensional particles.

The average value of the major diameters of the two-dimensional surfaces is 20 km or less, preferably 15 μm or less, and more preferably 10 μm or less from the viewpoint of dispersibility in the dispersion medium.

The major diameter of the two-dimensional surface refers to a major diameter when each MXene particle is approximated to an elliptical shape in an electron microscope micrograph, and the average value of the major diameters of the two-dimensional surfaces refers to a number average of the major diameter of 80 particles or more. As the electron microscope, a scanning electron microscope (SEM) photograph or a transmission electron microscope (TEM) photograph can be used.

The average value of the major diameters of the two-dimensional particles of the present embodiment may be measured by dissolving a material containing the two-dimensional particles in a solvent and dispersing the two-dimensional particles in the solvent. Alternatively, it may be measured from an SEM image of the material.

### (Average Value of Thicknesses of Two-Dimensional Particles)

The average value of the thicknesses of the two-dimensional particles of the present embodiment is preferably not less than 1 nm and not more than 15 nm. The thickness is preferably 10 nm or less, more preferably 7 nm or less, and still more preferably 5 nm or less. On the other hand, considering the thickness of the single-layer MXene particles, the lower limit of the thickness of the two-dimensional particles can be 1 nm.

The average value of the thicknesses of the two-dimensional particles is determined as a number average dimension (for example, a number average of at least 40) based on an atomic force microscope (AFM) photograph or a transmission electron microscope (TEM) photograph.

Hereinafter, a method for producing the two-dimensional particles is described in detail, but the present disclosure is not limited to such an embodiment.

The method for producing a two-dimensional particles comprises:
(a) providing a predetermined precursor;
(b) removing at least some A atoms from the precursor using an etching liquid and to obtain an etched product; and
(c) cleaning the etched product to obtain an etched and cleaned product.

The method may further comprise:
(d) mixing the etched and cleaned product with a metal compound containing a metal cation to obtain an intercalation product in which the metal cation is intercalated with the etched and cleaned product; and
(e) stirring the intercalated product to obtain a delaminated product in which the intercalated product is delaminated.

In one aspect, the etched product and the delaminated product can be used as the two-dimensional particles, and preferably the etched and cleaned product can be used as the two-dimensional particles.

Hereinafter, each step is described in detail.

### Step (a)

First, a predetermined precursor is prepared. The predetermined precursor that can be used in the present embodiment is a MAX phase that is a precursor of MXene, and represented by the following formula:

MₘAXₙ

wherein M is at least one metal of Group 3, 4, 5, 6, or 7, and includes at least Ti, X is a carbon atom, a nitrogen atom, or a combination thereof, A is at least one group 12, 13, 14, 15, or 16 element, n is not less than 1 and not more than 4, m is more than n and not more than 5.

The above M, X, n, and m are as described above.

A is at least one Group 12, 13, 14, 15, or 16 element, is usually a Group A element, typically Group IIIA and Group IVA, and more particularly may contain at least one selected from the group consisting of Al, Ga, In, Tl, Si, Ge, Sn, Pb, P, As, S, and Cd, and is preferably Al.

The MAX phase has a crystal structure in which a layer constituted by A atoms is located between two layers represented by MₘXₙ (each X may have a crystal lattice located in an octahedral array of M). When typically m=n+1, but not limited thereto, the MAX phase includes repeating units in which each one layer of X atoms is disposed in between adjacent layers of n+1 layers of M atoms (these are also collectively referred to as an “MₘXₙ layer”), and a layer of A atoms (“A atom layer”) is disposed as a layer next to the (n+1)th layer of M atoms. The A atom layer (and optionally a part of the M atoms) is removed by selectively etching (removing and optionally layer-separating) the A atoms (and optionally a part of the M atoms) from the MAX phase.

The MAX phase can be produced by a known method. For example, a TiC powder, a Ti powder, and an Al powder are mixed in a ball mill, and the resulting mixed powder is fired under an Ar atmosphere to obtain a fired body (block-shaped MAX phase). Thereafter, the fired body obtained is crushed by an end mill to obtain a powdery MAX phase for the next step.

### Step (b)

In a step (b), an etching treatment for removing at least a part of the A atoms from MₘAXₙ of the precursor by etching is performed using an etching liquid. As a result, a treated product in which at least a part of the layer composed of A atoms is removed is obtained while maintaining the layer represented by MₘXₙ in the precursor.

The etching liquid may contain an acid such as HF, HCl, HBr, HI, sulfuric acid, phosphoric acid, or nitric acid, and typically, an etching liquid containing an F atom can be used. Examples of the etching liquid include a mixed liquid of LiF and hydrochloric acid; a mixed liquid of hydrofluoric acid and hydrochloric acid; and a mixed liquid containing hydrofluoric acid, and these mixed liquids may further contain phosphoric acid or the like. The etching liquid can be typically an aqueous solution.

As the etching operation using the etching liquid and other conditions, conventionally performed conditions can be adopted.

### Step (c)

In a step (c), the treated product obtained by the etching treatment is cleaned to obtain an etched and cleaned product. By performing the cleaning, the acid and the like used in the etching treatment can be sufficiently removed.

The cleaning can be performed using a cleaning liquid, and typically, can be performed by mixing an etched product and a cleaning liquid. Such a cleaning liquid typically contains water, and pure water is preferable. On the other hand, a small amount of hydrochloric acid or the like may be further contained in addition to pure water. The amount of the cleaning liquid to be mixed with the etched product and the method for mixing the etched product and the cleaning liquid are not particularly limited. For example, as such a mixing method, stirring, centrifugation, and the like are performed in a state where the etched product and the cleaning liquid coexist. Examples of the stirring method include a stirring method using a handshake, an automatic shaker, a share mixer, a pot mill, or the like. The degree of stirring such as the stirring speed and the stirring time may be adjusted according to the amount, concentration, and the like of the etched product target to be treated. The cleaning with the cleaning liquid may be performed once or more, and it is preferable to perform the cleaning in the cleaning plural times. For example, specifically, the cleaning with a cleaning liquid may be performed by sequentially performing step (i) (to the treated product or the remaining precipitate obtained in the following (iii)), adding the cleaning liquid and stirring, step (ii) centrifuging the stirred product, and step (iii) discarding the supernatant liquid after centrifugation, and the steps (i) to (iii) may be repeated within a range of two times or more, for example, 15 times or less.

### Step (d)

In a step (d), an intercalation treatment of intercalating a metal cation is performed on the etched and cleaned product using a metal compound containing a metal cation to obtain an intercalated product. As a result, an intercalated product in which the metal cation is intercalated between two adjacent MₘXₙ layers is obtained. Such an intercalation treatment may be performed in a dispersion medium.

The metal cation may be the same as the metal cation contained in the two-dimensional particles, and may contain a Li cation or another metal cation.

However, the metal of the metal cation is different from the M atom. In addition, the metal of the metal cation is different from the A atom contained in the precursor.

Examples of the metal compound include an ionic compound in which the metal cation and the anion are bonded. Examples of the metal compound include a salt of the metal cation including an iodide, a phosphate, sulfide salt including a sulfate, a nitrate, an acetate, and a carboxylate. As the metal cation, a lithium ion is preferable, and as the metal compound, a metal compound containing a lithium ion is preferable, an ionic compound of a lithium ion is more preferable, and one or more of an iodide, a phosphate, and a sulfide salt of a lithium ion is further preferable. When a lithium ion is used as the metal ion, it is considered that water hydrated to the lithium ion has the most negative dielectric constant, and thus it is easy to form a single-layer.

A specific method of the intercalation treatment is not particularly limited, and for example, the etched and cleaned product and the metal compound may be mixed and stirred, or may be left to stand. The examples include stirring at room temperature. Examples of the stirring method include a method using a stirring bar such as a stirrer, a method using a stirring blade, a method using a mixer, a method using a centrifugal device, and the like, and the stirring time can be set according to the production scale of the single-layer/few-layer MXene particles, and can be set, for example, for 12 to 24 hours.

The intercalation treatment may be performed in the presence of a dispersion medium. Examples of the dispersion medium include water; and organic media such as N-methylpyrrolidone, N-methylformamide, N,N-dimethylformamide, methanol, ethanol, dimethylsulfoxide, ethylene glycol, and acetic acid.

The order of mixing the dispersion medium, the etched and cleaned product, and the metal compound is not particularly limited, but In one aspect, the metal compound may be mixed after mixing the dispersion medium and the etched and cleaned product. Typically, the etching liquid after the etching treatment may be used as the dispersion medium.

The intercalation treatment can be typically performed on the etched and cleaned product, but in another aspect, the precursor may be performed simultaneously with the etching treatment. Specifically, such an etching and intercalation treatment includes mixing a precursor, an etching liquid, and a metal compound containing a metal cation to remove at least some A atoms from the precursor, and intercalating the metal cation with the precursor from which the A atoms have been removed to obtain an intercalated product. As a result, at least a part of the A atoms is removed from the precursor (MAX), the MₘXₙ layer in the precursor remains, and an intercalated product in which the metal cation is intercalated between the plurality of adjacent MₘXₙ layers is obtained.

As the etching liquid and the metal compound used in the etching and intercalation treatment, the same etching liquid and metal compound as those used in the step (b) can be used, respectively.

### Step (e)

In a step (e), the intercalated product is stirred, and a delamination treatment for delaminating the intercalated product is performed to obtain a delaminated product. By such stirring, a shear stress is applied to the intercalated product, and at least a part between two adjacent MₘXₙ layers can be peeled off, and the MXene particles may become single-layered and few-layered.

Conditions for the delamination treatment are not particularly limited, and the delamination treatment can be performed by a known method. The example of the method of applying a shear stress to the intercalated product include dispersing the intercalated product in a dispersion medium and stirring the dispersion medium. Examples of the stirring method include stirring using a mechanical shaker, a vortex mixer, a homogenizer, ultrasonic treatment, a handshake, an automatic shaker, or the like. The degree of stirring such as the stirring speed and the stirring time may be adjusted according to the amount, concentration, and the like of the product target to be treated. For example, the slurry after the intercalation is centrifuged to discard the supernatant liquid, then pure water is added to the remaining precipitate, and stirring is performed by, for example, a handshake or an automatic shaker to perform layer separation. The removal of the unpeeled substance includes a step of performing centrifugal separation to discard the supernatant, and then cleaning the remaining precipitate with water. For example, (i) pure water is added to the remaining precipitate after discarding the supernatant and stirred, (ii) centrifugation is performed, and (iii) the supernatant liquid is recovered. This operation of (i) to (iii) is repeated one time or more, preferably two times or more and 10 times or less to obtain a supernatant liquid containing single-layer/few-layer MXene particles as a delaminated product. Alternatively, the supernatant liquid may be centrifuged, and the supernatant liquid after centrifugation may be discarded to obtain a clay containing single-layer/few-layer MXene particles as a delaminated product.

The delaminated product may be further cleaned. By such cleaning, at least a part of impurities and the like can be removed. Hereinafter, a treated product obtained by cleaning the delaminated product is also referred to as a delaminated cleaned product, and the delaminated cleaned product is included in the technical scope of the delaminated product.

In one aspect, the cleaning can be performed using a cleaning liquid, and typically, the cleaning can be performed by mixing the delaminated product and the cleaning liquid. In another aspect, the cleaning can be performed by acid-treating the delaminated product and then mixing the acid-treated product with a cleaning liquid. Examples of the acid include inorganic acids such as hydrochloric acid, sulfuric acid, nitric acid, phosphoric acid, perchloric acid, hydroiodic acid, hydrobromic acid, and hydrofluoric acid; organic acids such as acetic acid, citric acid, oxalic acid, benzoic acid, or sorbic acid, and these may be appropriately used. The concentration of the acid in the acid solution can be appropriately adjusted according to the delaminated product. In addition, the cleaning with a cleaning liquid may be performed by sequentially performing step (i) (to the treated product or the remaining precipitate obtained in the following (iii)), adding the cleaning liquid and stirring, step (ii) centrifuging the stirred product, and step (iii) discarding the supernatant liquid after centrifugation, and the steps (i) to (iii) may be repeated within a range of two times or more, for example, 15 times or less. The stirring can be performed using a handshake, an automatic shaker, a share mixer, a pot mill, or the like. The acid treatment may be performed one or more times, and if necessary, an operation of mixing with a fresh acid solution (acid solution not used for the acid treatment) and stirring may be performed within a range of two or more times, for example, 10 times or less. As the cleaning liquid, the same cleaning liquid as the cleaning liquid in the step (c) can be used. For example, specifically, water may be used as the cleaning liquid, and pure water is preferable. The mixing can be performed by the same method as the mixing method in the step (c), and specific examples thereof include stirring and centrifugation. Examples of the stirring method include a stirring method using a handshake, an automatic shaker, a share mixer, a pot mill, or the like.

The intermediate and the target product in the production method described above, for example, the intercalated product and the delaminated product may be dried by suction filtration, heat drying, freeze drying, vacuum drying, or the like.

The proportion of the two-dimensional particles in the membrane of the present disclosure may be preferably not less than 20 vol % and not more than 100 vol %, more preferably not less than 50 vol % and not more than 100 vol %, still more preferably not less than 70 vol % and not more than 100 vol %, still more preferably not less than 90 vol % and not more than 100 vol %, and still more preferably not less than 95 vol % and not more than 100 vol %.

The adsorbent material of the present disclosure may further contain one or more materials selected from a ceramics, a metal, and a resin in addition to the two-dimensional particles. For example, when the adsorbent material in the present embodiment contains a material such as a ceramics, a metal, or a resin, the adsorption can be readily performed with stability.

Examples of the ceramics include metal oxides such as silica, alumina, zirconia, titania, magnesia, cerium oxide, zinc oxide, barium titanate-based, hexaferrite, and mullite, and non-oxide ceramics such as silicon nitride, titanium nitride, aluminum nitride, silicon carbide, titanium carbide, tungsten carbide, boron carbide, and titanium boride.

Examples of the metal include metals of Groups 2 to 13 such as iron, titanium, magnesium, and aluminum; and an alloy based on the Group 2 to Group 13 metals.

The resin may be either a natural resin or a synthetic resin, and examples thereof include a hydrophilic resin and a hydrophobic resin. Such a hydrophilic resin may include a hydrophilic resin obtained by blending a hydrophilic auxiliary agent such as a surfactant into a hydrophobic resin to exhibit hydrophilicity, and a hydrophobic resin partially modified with a hydrophilic group.

Examples of the hydrophilic resin include a resin having a polar group (for example, a hydroxyl group, a substituted or unsubstituted amino group, a carboxyl group, a sulfonic acid group, a carbonyl group, an ester bond, an amide bond, or the like), and specific examples thereof include cellulose-based resins such as cellulose, cellulose acetate, and regenerated cellulose; polysulfone-based resins such as polysulfone and polyethersulfone; hydrophilic polyurethane; polyvinyl alcohol and copolymer thereof, sodium alginate; an acrylic resin; substituted or unsubstituted polyacrylamide; acrylonitrile; polyaniline sulfonic acid; and a polyamide resin such as nylon.

In one aspect, the hydrophilic resin is preferably a resin having a group capable of forming a hydrogen bond with a modifier or terminal T of the layer as the polar group. Examples of such a group include a carboxyl group, a sulfonic acid group, a carbonyl group, and an amide bond. Examples of the resin having these groups include water-soluble polyurethane, polyvinyl alcohol, sodium alginate, an acrylic acid-based water-soluble polymer, polyacrylamide, polyaniline sulfonic acid, and nylon, and among them, water-soluble polyurethane, polyvinyl alcohol, and sodium alginate are preferable, and water-soluble polyurethane is more preferable.

In another aspect, as the hydrophilic resin, an acrylic resin; polyacrylonitrile; cellulose-based resin; polysulfone-based resin; polyvinyl alcohol and copolymers thereof are preferable, polymethyl methacrylate, polyacrylonitrile, cellulose, cellulose acetate, polysulfone, polyvinyl alcohol, and copolymers of polyvinyl alcohol and polyethylene are more preferable, polysulfone, polymethyl methacrylate, and cellulose acetate are still more preferable, and polysulfone and polymethyl methacrylate are still more preferable. These resins can be used for dialysis, hemofiltration, and the like, and are suitable when the adsorbent material of the present disclosure is applied to a dialysis machine.

Examples of the hydrophobic resin include a resin not containing the polar group.

The adsorbent material of the present disclosure can be produced using the two-dimensional particles. The two-dimensional particle may be used as it is as the adsorbent material of the present disclosure, or the two-dimensional particles and one or more materials selected from a ceramics, a metal, and a resin to be used as necessary may be mixed and molded into a predetermined shape to form the adsorbent material.

In one aspect, when the adsorbent material contains a resin, the adsorbent material can be produced by mixing the two-dimensional particles and the resin to form a mixture and molding the mixture.

The mixing of the two-dimensional particles and the resin may be performed in the absence of a solvent or in the presence of a dispersion medium. Such a dispersion medium is typically water, and in some cases, other liquid substances may be contained in a relatively small amount (For example, 30% by mass or less, preferably 20% by mass or less based on the whole mass) in addition to water.

The two-dimensional particles and the resin can be stirred using a dispersing device such as a homogenizer, a propeller stirrer, a thin membrane swirling stirrer, a planetary mixer, a mechanical shaker, or a vortex mixer.

The molding method is not particularly limited, and for example, the mixture of the two-dimensional particles and the resin may be molded as it is into a predetermined shape, and the two-dimensional particles, the resin, and the dispersion medium may be mixed to obtain a mixture, such a mixture may be applied to a base material (for example, a substrate) to obtain a precursor membrane containing the dispersion medium, and at least a part of the dispersion medium contained in the precursor membrane may be removed by drying such a precursor membrane to form a membrane. Examples of the application method include a method of performing spray applying using a nozzle such as a one-fluid nozzle, a two-fluid nozzle, or an air brush, a method such as slit coating using a table coater, a comma coater, or a bar coater, screen printing, or metal mask printing, and an application method by spin coating, immersion, or dropping.

The application and drying may be repeated a plurality of times as necessary until a membrane having a desired thickness is obtained. The drying may be performed at a temperature of 400° C. or less using, for example, a normal pressure oven or a vacuum oven.

In another aspect, when the adsorbent material contains a ceramics or a metal, the adsorbent material can be produced by mixing the two-dimensional particles with, for example, a particulate ceramics or a metal and heating the mixture at a low temperature at which the composition of the two-dimensional particles can be maintained.

The adsorbent material is used for adsorbing a disease-causing substance, and can be used, for example, for adsorbing and removing or reducing the disease-causing substance in blood. The fact that the adsorbent material can adsorb the disease-causing substance can be confirmed, for example, in an adsorption test using human plasma.

The disease-causing substance preferably includes a protein. Examples of such a protein include uremic proteins (specifically, a medium-molecular-type uremic substance) such as parathyroid hormone, β2-microglobulin, and tumor necrosis factor (TNF)-α; cytokines (in one aspect, an inflammatory cytokine such as interleukin 18) such as interleukins, interferons, chemokines, hematopoietic factors, cell growth factors, and tumor necrosis factors; and other proteins such as albumin and M protein. The uremic protein (specifically, a medium-molecular-type uremic substance) corresponds to a uremic substance, and is expected to have a preventive effect on uremia by adsorbing the uremic substance. The inflammatory cytokine can cause various inflammatory symptoms in vivo, and an effect of preventing inflammation is expected by adsorbing them.

In the present disclosure, “uremic” means that the concentration in blood increases as the function of the kidney decreases, and can cause various symptoms associated with a renal disease such as an acute kidney disorder (including acute renal failure) and a chronic kidney disease (chronic renal failure, including end-stage renal failure), particularly various symptoms associated with a renal disorder.

In addition, by adsorbing an inflammatory substance as a disease-causing substance, the effect of preventing various symptoms associated with an inflammatory disease is expected. Various symptoms associated with an inflammatory disease include vascular dementia, inflammatory bowel disease, hepatitis, and myocarditis.

The disease-causing substance preferably contains an electrolyte (ionic substance). That is, the adsorbent material can also be used for adsorbing an electrolyte. Examples of such an electrolyte include a cation and an anion. Examples of such a cation include cations that can be excreted in the kidneys, and specifically include Na⁺, K⁺, Mg²⁺, and Ca²⁺. In the present disclosure, the anion includes an anion that can be excreted in the kidney, and specifically includes an anion containing P (for example, PO₄³⁻).

When the renal function decreases, the concentration of the electrolyte (in particular, an electrolyte which can be excreted in the kidney.) in the blood may increase, which may cause electrolyte imbalance such as hypernatremia, hyperkalemia, hypomagnesemia, and hyperphosphatemia. By adsorbing these electrolytes by the amount of the adsorbent material, the effect of preventing electrolyte imbalance is expected.

The disease-causing substance preferably further includes a protein-bound uremic substance such as creatinine, homocysteine, indoxyl sulfate, or p-cresyl sulfate; and a carcinogen. That is, the adsorbent can also be used for adsorbing a protein-bound uremic substance and a carcinogen. Therefore, by using the adsorbent material containing the two-dimensional particles as a separation membrane in a dialysis machine, the effect of treating and preventing the various diseases is expected.

The adsorbate of the present disclosure is expected to prevent or treat various symptoms associated with a renal disease such as an acute kidney disorder (including acute renal failure) and a chronic kidney disease (chronic renal failure, including end-stage renal failure), various symptoms associated with an inflammatory disease, particularly various symptoms associated with a renal disorder and/or an inflammatory disease, by being capable of adsorbing the adsorbent material. Specifically, It is expected to prevent or treat inflammatory diseases such as uremia; electrolyte imbalance such as hypernatremia, hyperkalemia, hypermagnesemia and hyperphosphatemia; autoimmune diseases; infectious diseases; inflammatory diseases; endocrine and metabolic diseases; circulatory disorders; blood diseases; gastrointestinal diseases; neuropathy; malignant tumors; drug intoxication; vascular dementia, inflammatory bowel disease, hepatitis, and myocarditis.

An adsorption method including adsorbing the disease-causing substance using the adsorbent material is also included in the technical scope of the present disclosure. Such an adsorption method preferably includes adsorbing a protein as a disease-causing substance using the adsorbent material. The adsorption method may further include adsorbing an electrolyte as a disease-causing substance. That is, the adsorption method may include adsorbing the protein and the electrolyte as the disease-causing substance using the adsorbent material. Such an adsorption method may further include adsorbing another adsorbate using the adsorbent material. In one aspect, the adsorption of the disease-causing substance can be performed by contacting the liquid containing an adsorbate such as a protein or an electrolyte as the disease-causing substance with the adsorbent material. The liquid containing such an adsorbate may contain substances other than the protein and the electrolyte as the disease-causing substances. Specific examples of the liquid containing the adsorbate include blood and a dialysate.

### Embodiment 2: Adsorbent Sheet

The adsorbent sheet in the present embodiment includes the adsorbent material. By containing the adsorbent material, such an adsorbent sheet can adsorb various adsorbates, particularly the disease-causing substance, and the adsorbent material can be physically fixed, so that adsorption performance can be stably exhibited.

The adsorbent sheet in the present embodiment may be made of the adsorbent material, and may further contain one or more materials selected from the ceramics, the metal, and the resin in addition to the adsorbent material. As the ceramics, the metal, and the resin, the same materials as the ceramic, the metal, and the resin that can be contained in the adsorbent material can be used, respectively.

When the adsorbent sheet in the present embodiment further contains one or more materials selected from the ceramics, the metal, and the resin, the adsorbent sheet may include a member in which the adsorbent material and the material are uniformly mixed, or may include a member containing the adsorbent material and a member containing the material, respectively.

In one aspect, the adsorbent sheet in the present embodiment includes a base material containing one selected from the above ceramics, a metal, and a resin; and a membrane containing the adsorbent material and disposed on the base material. The membrane containing such an adsorbent material may cover at least a part of the surface of the base material and may cover the entire surface of the base material.

The adsorbent sheet can be produced by forming a membrane containing an adsorbent material on a surface of the base material. The method for producing a membrane containing the adsorbent material is not particularly limited, and may include, for example, mixing the two-dimensional particles, a dispersion medium, and if necessary, two-dimensional particles to obtain a mixture, applying the mixture onto the base material to obtain a precursor membrane containing a dispersion medium, and drying the precursor membrane to remove at least a part of the dispersion medium contained in the precursor membrane to form a membrane. As a method of applying the mixture to the base material, for example, commonly used application methods such as immersion, brush, roller, roll coater, air spray, airless spray, curtain flow coater, roller curtain coater, die coater, and electrostatic coating can be used. The thickness of the adsorbent sheet and the thickness of the substrate can be appropriately set according to the application.

### Embodiment 3: Separation Membrane

The separation membrane in the present embodiment contains the adsorbent material. By containing the adsorbent material, such a separation membrane can adsorb various adsorbates, particularly the disease-causing substance, and the adsorbent material can be physically fixed, so that adsorption performance can be stably exhibited. The separation membrane in the present embodiment is particularly suitable for a separation membrane for artificial dialysis used for the hemodialysis or the like.

The separation membrane may be made of the adsorbent material, and may further contain one or more materials selected from the ceramics, the metal, and the resin in addition to the adsorbent material. In one aspect, it may include a base material and a base material containing one selected from the ceramics, the metal, and the resin; and a membrane containing the adsorbent material and disposed on the base material. The membrane containing such an adsorbent material may cover at least a part of the surface of the base material and may cover the entire surface of the base material. The shape of the base material is not particularly limited, and may be a film shape, a sheet shape, a plate shape, a porous shape, a hollow fiber shape, a bead shape, or the like.

As the ceramics, the metal, and the resin, the same materials as the ceramic, the metal, and the resin that can be contained in the adsorbent material can be used, respectively. Among them, cellulose-based and synthetic polymer-based materials used for hemodialysis and the like are preferable, and specifically, polymethyl methacrylate, polyacrylonitrile, cellulose, cellulose acetate, polysulfone, polyvinyl alcohol, vinyl alcohol copolymers such as copolymers of polyvinyl alcohol and ethylene, and the like can be preferably used, polysulfone, polymethyl methacrylate, and cellulose acetate are more preferably used, and polysulfone and polymethyl methacrylate are still more preferably used. The form of the separation membrane for artificial dialysis is not particularly limited, and examples thereof include a porous type, a hollow fiber type, and a flat membrane layered type.

### Embodiment 4: Artificial Dialysis Machine

The artificial dialysis machine according to the present embodiment includes the adsorbent material. By containing the adsorbent material, such an artificial dialysis machine can adsorb various adsorbates, particularly a disease-causing substance, and can be used for adsorption and removal of various waste products in hemodialysis, hemofiltration, hemodiafiltration, peritoneal dialysis, and the like. In other words, the adsorbent material of the present embodiment can be used in an artificial dialysis machine for performing the hemodialysis, hemofiltration, hemodiafiltration, peritoneal dialysis, and the like.

The artificial dialysis machine is classified into, for example, a hemodialysis machine and a peritoneal dialysis machine, and the hemodialysis machine is divided into a one-pass type (single-pass type) and a circulation type. Further, the circulation type includes by REDY systems (recirculating dialysate systems) and other systems. The artificial dialysis machine is also classified according to a method of removing urea without coming into contact with blood by a cross flow of blood and a dialysate from a patient and a method of directly filtering blood. In addition, a one-pass type peritoneal dialysis machine is mainly used. The adsorbent material of the present embodiment can be used for both of the hemodialysis and the peritoneal dialysis, and can be used as an adsorption membrane, a separation membrane, an adsorbent material cartridge, or the like in an artificial dialysis machine such as a hemodialysis machine or a peritoneal dialysis machine. For example, when used in a REDY system (recirculating dialysate system), the adsorbent material of the present embodiment may be used in the adsorbent material cartridge.

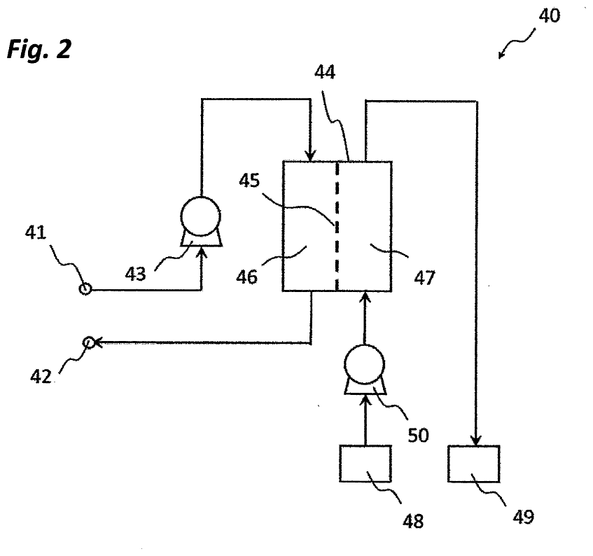

FIG. 2 schematically illustrates a one-pass type hemodialysis machine as an example of an artificial dialysis machine using the adsorbent material according to the present disclosure. In the hemodialysis machine 40 of FIG. 2, the blood before treatment introduced from the blood inlet 41 is fed to the blood purification machine 44 by the blood pump 43. On the other hand, the dialysate is fed from the unused dialysate tank 48 to the blood purification machine 44 by the dialysate pump 50. In the blood purification machine 44, the blood in the blood passage area 46 of the blood purification machine is subjected to hemodialysis, hemofiltration dialysis, or hemofiltration by the separation membrane 45, and the substance to be removed passes through the separation membrane 45 and moves to the dialysate passage area 47 of the blood purification machine. The purified blood is sent to the blood outlet 42. On the other hand, the dialysate in the dialysate passage area 47 containing the substance to be removed is fed to the used dialysate tank 49. Although not illustrated in FIG. 2, a device including a path for replenishing a drug, a protein, or the like to blood as necessary may be provided before treatment and/or during delivery of blood after treatment. In addition, a sensor for measuring the blood flow rate, the dialysate flow rate, and the protein concentration in the blood as necessary may be provided. An on-off valve capable of opening and closing the flow path may be provided in the middle of the flow path of the blood and/or the dialysate as necessary.

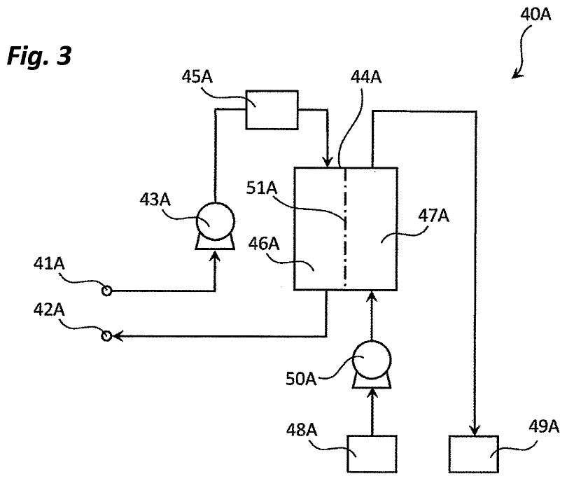

FIG. 3 schematically illustrates a one-pass type hemodialysis machine as another example of the artificial dialysis machine using the adsorbent according to the present disclosure. The hemodialysis machine 40A in FIG. 2 is different from the hemodialysis machine 40 in FIG. 2 in the aspects that a separation membrane 45A is disposed between a blood pump 43A and a blood purification machine 44A, and a semipermeable membrane 51A is disposed as a membrane for fractionating blood and a dialysate in the blood purification machine. The other configurations are the same as those of the hemodialysis machine 40 in FIG. 2, and are denoted by the same reference numerals, and description thereof is omitted.

In the hemodialysis machine 40A of FIG. 3, before blood passes through the blood purification machine 44A, the blood is contacted with the separation membrane 45A so that an adsorbate such as a disease-causing substance can be adsorbed, and therefore dialysis efficiency can be improved.

The separation membrane 45A may be cladded with a resin or the like and disposed in the form of a column. In the column, a filter may be disposed on each of the blood inflow side and the blood outflow side of the column so that blood is distributed in the separation membrane 45A.

In the hemodialysis machine 40A of FIG. 3, the membrane for fractionating blood and dialysate is a semipermeable membrane 51A, but the separation membrane of the present disclosure may be used instead of the semipermeable membrane 51A.

The present disclosure is described more specifically with reference to the following Examples, but the present disclosure is not limited thereto.

### Example 1

### [Production of MXene]

In Examples 1 and 2, (1) preparation of the precursor (MAX), (2) etching of the precursor, and (3) cleaning and drying described in detail below were sequentially performed to prepare two-dimensional particles.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The resulting mixed powder was fired at 1,350° C. for 2 hours under an Ar atmosphere. The fired body (block) thus obtained was crushed with an end mill to a maximum size of 40 μm or less. In this way, Ti₃AlC₂ particles were obtained as a precursor (MAX).

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (Sieving with 45 μm mesh)
Etching liquid composition: 50 mass % HF 6 mL,
   H₂O 18 mL
   HCl (12M) 36 mL
Precursor charge amount: 3.0 g
Etching container: 100 mL Iboy
Etching temperature: 35° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3,500 G using a centrifuge, and then the supernatant liquid was discarded. An operation of adding 40 mL of pure water to each centrifuge tube, centrifuging again at 3,500 G, and separating and removing the supernatant liquid was repeated 11 times. After final centrifugation, the supernatant liquid was discarded to obtain a Ti₃C₂Tₛ-water medium clay. The obtained clay was dried by freeze drying to obtain a dried powder of two-dimensional particles.

### Example 2

In Example 2, (1) the precursor (MAX) was prepared in the same manner as in Example 1, then the following step (2) was performed, and (3) cleaning and drying were performed in the same manner as in Example 1 to prepare two-dimensional particles.

### (1) Preparation of Precursor: Same as in Example 1

### (2) Precursor Etching

Precursor: Ti₃AlC₂ (Sieving with 45 μm mesh)
Etching liquid composition: 50 mass % HF 5 mL,
   H₂O 45 mL
Precursor charge amount: 3.0 g
Etching container: 100 mL Iboy
Etching temperature: 35° C.
Etching time: 24 h
Stirrer rotation speed: 400 rpm

### (3) Cleaning: Same as in Example 1

### Example 3

In Example 3, (1) the precursor (MAX) was prepared in the same manner as in Example 1, then the following step (2) was performed, and (3) cleaning and drying were performed in the same manner as in Example 1 to prepare two-dimensional particles.

### (1) Preparation of Precursor: Same as in Example 1

### (2) Precursor Etching

Precursor: Ti₃AlC₂ (Sieving with 45 μm mesh)
Etching liquid composition: 49 mass % HF aqueous solution 6 mL,
   H₂O 9 mL
   85 mass % H₃PO₄ aqueous solution 45 mL
Precursor charge amount: 3.0 g
Etching container: 100 mL Iboy
Etching temperature: 35° C.
Etching time: 24 h
Stirrer rotation speed: 400 rpm

### (3) Cleaning: Same as in Example 1

### Example 4

In Example 4, (1) the precursor (MAX) was prepared in the same manner as in Example 1, then the following step (2) was performed, and (3) cleaning and drying were performed in the same manner as in Example 1 to prepare two-dimensional particles.

### (1) Preparation of Precursor: Same as in Example 1

### (2) Precursor Etching

Precursor: Ti₃AlC₂ (Sieving with 45 μm mesh)
Etching liquid composition: LiF 4.8 g
   HCl (9M) 60 mL
Precursor charge amount: 3.0 g
Etching container: 100 mL Iboy
Etching temperature: 35° C.
Etching time: 24 h
Stirrer rotation speed: 400 rpm

### (3) Cleaning: Same as in Example 1

### Example 5

In Example 5, (1) preparation of the precursor (MAX), (2) etching of the precursor, and (3) cleaning and drying described in detail below were sequentially performed to prepare two-dimensional particles.

### (1) Preparation of Precursor (MAX)

Into a ball mill containing a zirconia ball, 73 g of Ti powder, 47.2 g of TiN powder, 20.6 g of Al powder, and 9.2 g of C powder (all manufactured by Kojundo Chemical Laboratory Co., Ltd.) were charged, and mixed for 24 hours. The resulting mixed powder was fired at 1,400° C. for 2 hours under an Ar atmosphere. The fired body (block) thus obtained was crushed with an end mill to a maximum size of 40 μm or less. In this way, Ti₃AlCN particles were obtained as a precursor (MAX).

### (2) Etching of Precursor

Using the Ti₃AlCN particles (powder) prepared by the above method, etching was performed under the following etching conditions to obtain a solid-liquid mixture (slurry) containing a solid component derived from the Ti₃AlCN powder.

### (Etching Conditions)

Precursor: Ti₃AlCN (Sieving with 45 μm mesh)
Etching liquid composition: 48 mass % HF 6 mL
   38.8 mass % HCl 36 mL
   H₂O 18 mL
Precursor charge amount: 3.0 g
Etching container: 100 mL Iboy
Etching temperature: 25° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3,500 G using a centrifuge, and then the supernatant liquid was discarded. An operation of adding 40 mL of pure water to each centrifuge tube, centrifuging again at 3,500 G, and separating and removing the supernatant liquid was repeated 11 times. After final centrifugation, the supernatant liquid was discarded to obtain a Ti₃CNTₛ-water medium clay. The obtained clay was dried by freeze drying to obtain a dried powder of two-dimensional particles.

### Example 6

In Example 6, (1) preparation of the precursor (MAX), (2) etching of the precursor, and (3) cleaning and drying described in detail below were sequentially performed to prepare two-dimensional particles.

### (1) Preparation of Precursor (MAX)

Into a ball mill containing zirconia balls, 21.5 g of the V powder, 6.3 g of the Al powder, and 2.3 g of the C powder (all manufactured by Kojundo Chemical Laboratory Co., Ltd.) were charged, and mixed for 24 hours. The resulting mixed powder was fired at 1,550° C. for 2 hours under an Ar atmosphere. The fired body (block) thus obtained was crushed with a jaw crusher to a maximum dimension of 45 μm or less. In this way, V₂AlC particles were obtained as a precursor (MAX).

### (2) Etching of Precursor

Using the V₂AlC particles (powder) prepared by the above method, etching was performed under the following etching conditions to obtain a solid-liquid mixture (slurry) containing a solid component derived from the V₂AlC powder.

### (Etching Conditions)

Precursor: V₂AlC (Sieving with 45 μm mesh)
Etching liquid composition: 48 mass % HF 6 mL
   38.8 mass % HCl 24 mL
Precursor charge amount: 3.0 g
Etching container: 100 mL Iboy
Etching temperature: 50° C.
Etching time: 48 h
Stirrer rotation speed: 400 rpm

### (3) Cleaning and Drying

The slurry was divided into two portions, each of which was inserted into two 50 mL centrifuge tubes, centrifuged under the condition of 3,500 G using a centrifuge, and then the supernatant liquid was discarded. An operation of adding 40 mL of pure water to each centrifuge tube, centrifuging again at 3,500 G, and separating and removing the supernatant liquid was repeated 11 times. After final centrifugation, the supernatant liquid was discarded to obtain a V₂CTₛ-water medium clay. The obtained clay was dried by freeze drying to obtain a dried powder of two-dimensional particles.

### Example 7

In Example 7, (1) preparation of the precursor (MAX), (2) etching of the precursor, and (3) cleaning and drying described in detail below were sequentially performed to prepare two-dimensional particles.

### (1) Preparation of Precursor (MAX)

In a ball mill containing zirconia balls, 105.4 g of Ti powder, 32.7 g of Al powder, and 11.9 g of C powder (all manufactured by Kojundo Chemical Laboratory Co., Ltd.) were charged, and mixed for 24 hours. The resulting mixed powder was fired at 1,550° C. for 2 hours under an Ar atmosphere. The fired body (block) thus obtained was crushed with an end mill to a maximum size of 40 μm or less. In this way, Ti₂AlC particles were obtained as a precursor (MAX).

### (2) Etching of Precursor

Using the Ti₂AlC particles (powder) prepared by the above method, etching was performed under the following etching conditions to obtain a solid-liquid mixture (slurry) containing a solid component derived from the Ti₂AlC powder.

### (Etching Conditions)

Precursor: Ti₂AlC (Sieving with 45 μm mesh)
Etching liquid composition: 48 mass % HF 2 mL
   38.8 mass % HCl 12 mL
   H₂O 6 mL
Precursor charge amount: 1.0 g
Etching container: 100 mL Iboy
Etching temperature: 35° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3,500 G using a centrifuge, and then the supernatant liquid was discarded. An operation of adding 40 mL of pure water to each centrifuge tube, centrifuging again at 3,500 G, and separating and removing the supernatant liquid was repeated 11 times. After final centrifugation, the supernatant liquid was discarded to obtain a Ti₂CTₛ-water medium clay. The obtained clay was dried by freeze drying to obtain a dried powder of two-dimensional particles.

### Example 8

In Example 8, (1) preparation of the precursor (MAX), (2) etching of the precursor, and (3) cleaning and drying described in detail below were sequentially performed to prepare two-dimensional particles.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1350° C. for 2 hours. The fired body (block) thus obtained was crushed with an end mill to a maximum size of 40 μm or less. As a result, Ti₃AlC₂ particles were obtained as MAX particles.

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (Sieving with 45 μm mesh)
Etching liquid composition: 48% HF 6 mL
   H₂O 54 mL
Precursor charge amount: 3.0 g
Etching container: 100 mL Iboy
Etching temperature: 50° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3500 G using a centrifuge, and then the supernatant liquid was discarded. An operation of adding 40 mL of pure water to the remaining precipitate in each centrifuge tube, centrifuging again at 3500 G, and separating and removing the supernatant liquid was repeated 11 times. After final centrifugation, the supernatant liquid was discarded to obtain a Ti₃C₂Tₓ-water medium clay. The obtained clay was dried by freeze drying to obtain a dried powder of two-dimensional particles.

### Example 9

In Example 9, (1) preparation of the precursor (MAX), (2) etching of the precursor, and (3) cleaning and drying described in detail below were sequentially performed to prepare two-dimensional particles.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1350° C. for 2 hours. The fired body (block) thus obtained was crushed with an end mill to a maximum size of 40 μm or less. As a result, Ti₃AlC₂ particles were obtained as MAX particles.

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (Sieving with 45 μm mesh)
Etching liquid composition: 48 mass % HF 6 mL
   38.8 mass % HCl 38 mL
   H₂O 16 mL
Precursor charge amount: 3.0 g
Etching container: 100 mL Iboy
Etching temperature: 50° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3500 G using a centrifuge, and then the supernatant liquid was discarded. An operation of adding 40 mL of pure water to the remaining precipitate in each centrifuge tube, centrifuging again at 3500 G, and separating and removing the supernatant liquid was repeated 11 times. After final centrifugation, the supernatant liquid was discarded to obtain a Ti₃C₂Tₓ-water medium clay. The obtained clay was dried by freeze drying to obtain a dried powder of two-dimensional particles.

### Example 10

In Example 10, (1) preparation of a precursor (MAX), (2) etching of the precursor, (3) cleaning, (4) Li intercalation, (5) delamination, (6) modification with an amino group, and drying described in detail below were sequentially performed to prepare two-dimensional particles.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was calcined in an Ar atmosphere at 1350° C. for 2 hours. The fired body (block) thus obtained was crushed with an end mill to a maximum size of 40 μm or less. As a result, Ti₃AlC₂ particles were obtained as MAX particles.

### (2) Etching of Precursor

Using the Ti₃AlC₂ particles (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (Sieving with 45 μm mesh)
Etching liquid composition: 48 mass % HF 2 mL
   38.8 mass % HCl 38 mL
   H₂O 16 mL
Precursor charge amount: 3.0 g
Etching container: 100 mL Iboy
Etching temperature: 50° C.
Etching time: 24 h
Stirrer rotation speed: 400 rpm

### (3) Cleaning and Drying

The slurry was divided into two portions, each of which was inserted into two 50 mL centrifuge tubes, centrifuged under the condition of 3500 G using a centrifuge, and then the supernatant liquid was discarded. An operation of adding 40 mL of pure water to the remaining precipitate in each centrifuge tube, centrifuging again at 3500 G, and separating and removing the supernatant liquid was repeated 11 times. After final centrifugation, the supernatant liquid was discarded to obtain a Ti₃C₂Tₓ-water medium clay.

### (4) Li Intercalation

The Ti₃C₂Tₓ-water medium clay prepared by the above method was stirred at not less than 20° C. and not more than 25° C. for 12 hours using LiCl as a Li-containing compound to perform Li intercalation according to the following conditions.

### (Conditions of Li Intercalation)

Ti₃C₂Tₓ-water medium clay (MXene after cleaning): Solid content 0.75 g
LiCl: 0.75 g
Intercalation container: 100 mL Iboy
Temperature: Not less than 20° C. and not more than 25° C. (room temperature)
Time: 10 h
Stirrer rotation speed: 800 rpm

### (5) Delamination

(i) To the Ti₃C₂Tₓ-water medium clay, 40 mL of pure water was added, then the mixture was stirred with a shaker for 15 minutes, (ii) the mixture was centrifuged at 3500 G, and (iii) the supernatant liquid was recovered as a single-layer MXene-containing liquid. The operations (i) to (iii) were repeated four times in total to obtain a single-layer MXene-containing supernatant liquid. Further, this supernatant liquid was centrifuged under the conditions of 4,300 G and 2 hours using a centrifuge, and then the supernatant liquid was discarded to obtain a single-layer/few-layer MXene-containing clay as a single-layer/few-layer MXene-containing sample.

(6) Modification with Amino Group and Drying

To 0.34 g of single-layer/few-layer MXene-containing clay, 20 mg of 6-aminohexyl phosphate hydrochloride and 5 mL of pure water were added, and the mixture was stirred at room temperature at a stirrer rotation speed of 800 rpm for 2 hours. The obtained slurry was centrifuged at a rotation speed of 8,000 rpm for 8 minutes, and the supernatant liquid was discarded. Pure water having the same volume as the discarded supernatant liquid was added, and centrifuged at a rotation speed of 8,000 rpm for 8 minutes. Discard of the supernatant liquid, addition of pure water having the same volume as that of the discarded supernatant liquid, and centrifugation at a rotation speed of 8,000 rpm for 8 minutes were repeated once. Subsequently, the supernatant liquid was discarded, and ethanol having the same volume as that of the discarded supernatant liquid was added and dispersed, and then naturally dried to obtain Ti₃C₂ particles (powder) modified with an amino group.

### Comparative Example 1

The spherical adsorption charcoal (“KREMEZIN Tablets 500 mg”, manufactured by KUREHA CORPORATION) was powdered using a mortar and subjected to adsorption evaluation.

### Comparative Example 2

An adsorption type blood purifier (“Lixelle”, manufactured by KANEKA CORPORATION) was disassembled, and the extracted adsorbing body was subjected to adsorption evaluation.

### Comparative Example 3

A medicinal charcoal (manufactured by Nichi-Iko Pharmaceutical Co., Ltd.) was subjected to adsorption evaluation.

### Adsorption Evaluation Method

Human plasma collected from a healthy subject, two-dimensional particles of Example, and spherical adsorption charcoal, adsorption type blood purifier, or medicinal charcoal of Comparative Example (hereinafter, these are collectively referred to as “adsorbent material”) were weighed in a 50 mL centrifuge tube, and shaken and stirred for 60 minutes using a constant temperature shaker (TAITEC BR-33FL) set at 37° C. Thereafter, several mL of the mixed solution was sampled, the adsorbent material was separated with a syringe filter (Millex diameter for Merck Millipore sterilization: 33 mm, hole diameter: 0.45 μm) having a hole diameter of 0.45 μm, and then component analysis was performed. In Examples 1 to 4 and Comparative Example 1 to 3, the human plasma amount was 10 mL and the adsorbent material amount was 0.6 g, in Example 5 to 8 and Example 10, the human plasma amount was 4 mL and the adsorbent material amount was 0.2 g, and in Example 9, the human plasma amount was 4 mL and the adsorbent material amount was 0.2 g.

In Examples 1 to 4 and Comparative Examples 1 to 3, component analysis was performed for Na, P, K, Urea, Creatinine, Homocysteine, Folic acid, Parathyroid hormone (Prathyroid hormone), β2-microglobulin (β2-microgloblin), Interleukin-18, Trypsin, TNF-α (Tumor necrosis factor-α), α-amylase, and Albumin.

In Examples 5 to 10, component analysis was performed for Albumin, Chlor (Cl), serum urea nitrogen (BUN), α-amylase (AMY), lipase, Na, K, inorganic phosphorus (IP), and β2-microglobulin.

The measurement of Na, K and Cl was performed by an electrode method; the measurement of P, inorganic phosphorus, α-amylase, lipase, and creatinine was performed by an enzymatic method; the measurement of urea and serum urea nitrogen was performed by a urease/GLDH/UV method; the measurement of homocysteine was performed by HPLC method; the measurement of folic acid was performed by the CLIA method; the measurement of parathyroid hormone was performed by the ECLIA method; the measurement of β2-microglobulin was performed by a latex agglutination method; the measurement of interleukin-18, trypsin, and TNF-α was performed by an EIA method; the measurement of albumin was performed by a colorimetric method (BGC method).

Note that the same treatment was also performed on the sample containing no adsorbent material and containing only human plasma in the centrifuge tube, and the sample was used as a baseline of component concentration. The concentration of the component A contained in the human plasma after the adsorption test was performed was defined as C_{A1}, and the concentration of the component A at the baseline was defined as C_{A0}, and the adsorption removal rate was calculated based on the following formula. When the adsorption removal rate was less than 0% by mass, the adsorption removal rate was set to 0% by mass.

$$Adsorption removal rate ( mass \% ) = ( {C}_{A 0} - {C}_{A 1} ) / {C}_{A 0} \times 100$$

The results are shown in Tables 1 and 2.

TABLE 1

| | molecular | Example | Example | Example | Example | Comparative | Comparative | Comparative |
|---|---|---|---|---|---|---|---|---|
| Components | weight | 1 | 2 | 3 | 4 | Example 1 | Example 2 | Example 3 |
| Na | 23 | 21 | 22 | 21 | 20 | 0 | 0 | 0 |
| P | 31 | 44 | 46 | 40 | 42 | 0 | 0 | 0 |
| K | 39 | 29 | 29 | 29 | 28 | 0 | 0 | 0 |
| Urea | 60 | 0 | 0 | 2 | 1 | 13 | 0 | 9 |
| Creatinine | 113 | 92 | 91 | 90 | 90 | 91 | 0 | 90 |
| Homocysteine | 135 | 70 | 72 | 68 | 70 | 0 | 0 | 86 |
| Folic acid | 441 | 87 | 80 | 81 | 75 | 68 | 0 | 42 |
| Parathyroid | 9,500 | 73 | 75 | 70 | 70 | 0 | 0 | 35 |
| hormone | | | | | | | | |
| β2-microglobulin | 11,818 | 50 | 55 | 46 | 52 | 0 | 99 | 5 |
| Interleukin-18 | 18,000 | 29 | 33 | 28 | 27 | 0 | 0 | 0 |
| Trypsin | 24,000 | 26 | 25 | 22 | 24 | 0 | 0 | 0 |
| Tumor necrosis | 25,000 | 50 | 39 | 28 | 32 | 0 | 0 | 0 |
| factor-α | | | | | | | | |
| α-amylase | 54,000 | 26 | 22 | 22 | 18 | 0 | 0 | 0 |
| Albumin | 66,000 | 11 | 10 | 8 | 11 | 0 | 0 | 0 |

TABLE 2

| | molecular | Example | Example | Example | Example | Example | Example |
|---|---|---|---|---|---|---|---|
| Components | weight | 5 | 6 | 7 | 8 | 9 | 10 |
| Albumin | 66,000 | 3 | 3 | 0 | 0 | 3 | 0 |
| Cl | 35 | 95 | 13 | 95 | 55 | 30 | 0 |
| BUN | 60 | 0 | 0 | 0 | 0 | 0 | 0 |
| AMY | 54,000 | 0 | 0 | 16 | 0 | 0 | 0 |
| Lipase | 45,000 | 0 | 0 | 0 | 0 | 0 | 0 |
| Na | 23 | 19 | 6 | 26 | 6 | 8 | 1 |
| K | 39 | 32 | 21 | 46 | 10 | 11 | 3 |
| IP | 31 | 56 | 0 | 58 | 7 | 42 | 33 |
| β2-microglobulin | 11,818 | 8 | 0 | 75 | 0 | 0 | 25 |

It was confirmed that the adsorbent materials of Examples 1 to 4 adsorb, as disease-causing substances, middle molecular type uremic substances such as parathyroid hormone, β2-microglobulin, and tumor necrosis factor (TNF)-α; inflammatory cytokines such as interleukin-18; and proteins such as albumin, and further exhibit the ability to adsorb electrolytes such as Na⁺, K⁺, and P; the protein-bound uremic substances such as creatinine and homocysteine. Therefore, the adsorbent material containing the two-dimensional particles can adsorb various adsorbates, and application to an adsorbent sheet, a separation membrane, and an artificial dialysis machine using such an adsorbent material is expected.

In addition, it was confirmed that the two-dimensional particles of Examples 5 to 10 exhibited an action of adsorbing a disease-causing substance, particularly an electrolyte (ionic substance), a uremic substance, and other proteins. Therefore, the adsorbent material containing the two-dimensional particles can adsorb various adsorbates, and application to an adsorbent sheet, a separation membrane, and an artificial dialysis machine using such an adsorbent material is expected.

In Comparative Examples 1 to 3, predetermined two-dimensional particles were not contained, and as a result of the adsorption test, it was confirmed that the adsorbate was limited. In particular, it was not confirmed that any of the adsorbent materials of Comparative Examples 1 to 3 adsorbed an electrolyte such as Na⁺, K⁺, or P.

The present disclosure comprises the following:

[1] An adsorbent material used for adsorbing a disease-causing substance, the adsorbent material comprising two-dimensional particles having one or plural layers, the one or plural layers comprising a layer body represented by:

MₘXₙ

wherein M is at least one metal of Group 3, 4, 5, 6, or 7, X is a carbon atom, a nitrogen atom, or a combination thereof, n is not less than 1 and not more than 4, m is more than n but not more than 5, and a modifier or terminal T exists on a surface of the layer body, wherein T is at least one selected from a hydroxyl group, a fluorine atom, a chlorine atom, an oxygen atom, and a hydrogen atom.

[2] The adsorbent material according to [1], wherein the disease-causing substance contains a protein.

[3] The adsorbent material according to [1] or [2], wherein the disease-causing substance contains a uremic substance having a molecular weight of 100 or more.

[4] The adsorbent material of [3], wherein the uremic substance comprises β2-microglobulin.

[5] The adsorbent material according to any one of [1] to [4], wherein the disease-causing substance contains a cytokine.

[6] The adsorbent material according to [5], wherein the cytokine comprises at least one selected from interleukins, interferons, a chemokine, a hematopoietic factor, a cell growth factor, and a tumor necrosis factor.

[7] The adsorbent material according to any one of [1] to [6], wherein the disease-causing substance contains an electrolyte.

[8] The adsorbent material according to [7], wherein the electrolyte contains at least one selected from Na⁺, K⁺, Mg²⁺, Ca²⁺, and PO₄³⁻.

[9] An adsorbent sheet comprising the adsorbent material according to any one of [1] to [8].

[10] A separation membrane comprising the adsorbent material according to any one of [1] to [8].

[11] An artificial dialysis machine comprising the adsorbent material according to any one of [1] to [8].

[12] A method for producing an adsorbent material used for adsorption of a disease-causing substance, the method comprising:
(a) providing a precursor represented by:

MₘAXₙ
wherein M is at least one metal of Group 3, 4, 5, 6, or 7, and includes at least Ti, X is a carbon atom, a nitrogen atom, or a combination thereof, A is at least one group 12, 13, 14, 15, or 16 element, n is not less than 1 and not more than 4, m is more than n and not more than 5;
(b) removing at least some A atoms from the precursor using an etching liquid to obtain an etched product;
(c) cleaning the etched product to obtain an etched and cleaned product including two-dimensional particles.

### REFERENCE SIGNS LIST

1a, 1b Layer body (MₘXₙ layer)
3a, 5a, 3b, 5b Modifier or terminal T
7a, 7b MXene layer
10a, 10b MXene particles (particles of layered material)
40 Hemodialysis machine
41, 41A Blood inlet
42, 42A Blood outlet
43, 43A Blood pump
44, 44A Blood purification machine
45, 45A Separation membrane
46, 46A Blood passage area of blood purification machine
47, 47A Dialysate passage area of blood purification machine
48, 48A Unused dialysate tank
49, 49A Used dialysate tank
50, 50A Dialysate pump
51 Semipermeable membrane

1. An adsorbent material used for adsorbing a disease-causing substance, the adsorbent material comprising:
two-dimensional particles having one or plural layers, the one or plural layers comprising a layer body represented by:
MₘXₙ
wherein
M is at least one metal of Group 3, 4, 5, 6, or 7,
X is a carbon atom, a nitrogen atom, or a combination thereof,
n is not less than 1 and not more than 4,
m is more than n but not more than 5, and
a modifier or terminal T exists on a surface of the layer body,
wherein T is at least one selected from a hydroxyl group, a fluorine atom, a chlorine atom, an oxygen atom, and a hydrogen atom,
wherein the disease-causing substance comprises one or more selected from β2-microglobulin or an electrolyte, and
wherein the electrolyte comprises one or more selected from Na⁺, K⁺, Mg²⁺, Ca²⁺, or PO₄³⁻.

2. The adsorbent material according to claim 1, wherein a ratio of (an average value of a major diameter of two-dimensional surfaces of the two-dimensional particles)/(an average value of thicknesses of the two-dimensional particles) is 1.2 or more.

3. The adsorbent material according to claim 1, wherein an average flake size of the two-dimensional particles is not less than 0.01 μm and not more than 20 μm.

4. The adsorbent material according to claim 1, wherein an average value of thicknesses of the two-dimensional particles is not less than 1 nm and not more than 15 nm.

5. The adsorbent material according to claim 1, wherein the disease-causing substance contains a protein.

6. The adsorbent material according to claim 1, wherein the disease-causing substance contains a cytokine.

7. The adsorbent material according to claim 6, wherein the cytokine comprises at least one of interleukins, interferons, a chemokine, a hematopoietic factor, a cell growth factor, and a tumor necrosis factor.

8. The adsorbent material according to claim 1, further comprising one or more materials selected from a ceramic, a metal, and a resin.

9. An adsorbent sheet comprising the adsorbent material according to claim 1.

10. A separation membrane comprising the adsorbent material according to claim 1.

11. An artificial dialysis machine comprising the adsorbent material according to claim 1.

12. A method for producing an adsorbent material used for adsorption of a disease-causing substance, the method comprising:
(a) providing a precursor represented by the following:
MₘAXₙ
wherein
M is at least one metal of Group 3, 4, 5, 6, or 7, and includes at least Ti,
X is a carbon atom, a nitrogen atom, or a combination thereof,
A is at least one group 12, 13, 14, 15, or 16 element,
n is not less than 1 and not more than 4, and
m is more than n and not more than 5;
(b) removing at least some A atoms from the precursor using an etching liquid to obtain an etched product; and
(c) cleaning the etched product to obtain an etched and cleaned product including two-dimensional particles,
wherein the disease-causing substance comprises one or more selected from β2-microglobulin or electrolyte, and
wherein the electrolyte comprises one or more selected from Na⁺, K⁺, Mg²⁺, Ca²⁺, or PO₄³⁻.

13. The method according to claim 12, further comprising (d) mixing the etched and cleaned product with a metal compound containing a metal cation to obtain an intercalation product in which the metal cation is intercalated with the etched and cleaned product.

14. The method according to claim 13, further comprising (e) stirring the intercalated product to obtain a delaminated product in which the intercalated product is delaminated.

15. The method according to claim 12, further comprising mixing the two-dimensional particles and one or more materials selected from a ceramic, a metal, and a resin to form the adsorbent material.

16. An adsorbent material used for adsorbing a disease-causing substance, the adsorbent material comprising:
MXene,
wherein the disease-causing substance comprises one or more selected from β2-microglobulin or an electrolyte, and
wherein the electrolyte comprises one or more selected from Na⁺, K⁺, Mg²⁺, Ca²⁺, or PO₄³⁻.
